(19) United States
(12) Patent Application Publication
(10) Pub. No.: US 2026/0227474 A1
(43) Pub. Date: Aug. 6, 2026

(54) SYSTEM AND METHOD FOR WATER, FAT, AND FIELD INHOMOGENEITY MAGNETIC RESONANCE IMAGING FROM SINGLE MAGNETIC RESONANCE IMAGE ACQUISITION

(71) Applicant: GE Precision Healthcare LLC, Waukesha, WI (US)

(72) Inventors: Hongxu Yang, Helmond (NL); Harsh Kumar Agarwal, Jaipur (IN); Ramesh Venkatesan, Bangalore (IN); Lehel Mihály Ferenczi, Budapest (HU); Gopal Biligeri Avinash, Concord, CA (US); Uday Damodar Patil, Bangalore (IN)

(21) Appl. No.: 19/045,012

(22) Filed: Feb. 4, 2025

(51) Int. Cl. G01R 33/48; G01R 33/56; G01R 33/561; G01R 33/565; G06T 7/00

(52) U.S. Cl. CPC G01R 33/4828; G01R 33/5608; G01R 33/5615; G01R 33/56527; G06T 7/0012; G06T 2207/10088; G06T 2207/20081

(57) ABSTRACT

A method for generating magnetic resonance images includes obtaining, via a processing system, a single complex magnetic resonance image per slice from magnetic resonance scan data acquired of a subject with a magnetic resonance scanner utilizing a chemical shifted sequence where signal contributions from different pre-defined components to acquired echo signals are separated, wherein the single complex magnetic resonance image comprises a non-zero phase angle between the different pre-defined components. The method also includes inputting, via the processing system, the single complex magnetic resonance image for each slice into a decomposition model trained to predict chemical shifted-like images from the single complex magnetic resonance image for each slice. The method further includes outputting, via the processing system, from the decomposition model the chemical shifted-like images of one or more pre-defined components of the different pre-defined components based on the single complex magnetic resonance image for each slice.

### BACKGROUND

The subject matter disclosed herein relates to medical imaging and, more particularly, to systems and methods for water, fat, and field inhomogeneity magnetic resonance imaging (MRI) from single magnetic resonance image acquisition.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During MRI, when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

Water and fat separation can generate several key MRI contrasts. Fat suppressed proton density weighted MRI or water only proton density weighted MRI is primary contrast for musculoskeletal imaging such as knee and shoulder. The Dixon based MRI method is used to acquire fat suppressed MRI images when it may be challenging to utilize other methods for acquiring fat suppressed MRI. It may be challenging due to main field inhomogeneity and presence of implants. However, Dixon based MRI method is associated with acquired multiple MR images of a same anatomy with different complex additions of water and fat which increases the scan time and/or reduces the signal-to-noise ratio (SNR) per unit scan time.

In addition, existing image to image translation artificial intelligence (AI) models use real-valued images (e.g., in-phase images) to predict the water or fat. Thus, the image information is only in real-space while ignoring the phase information as the deep prior. These models tend to over predict as the phase angle zero degrees so there is no ability to separate water and fat for anything other than prior information.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for generating magnetic resonance images is provided. The computer-implemented method includes obtaining, via a processing system including one or more processors, a single complex magnetic resonance image per slice from magnetic resonance scan data acquired of a subject with a magnetic resonance scanner utilizing a chemical shifted sequence where signal contributions from different pre-defined components to acquired echo signals are separated, wherein the single complex magnetic resonance image includes a non-zero phase angle between the different pre-defined components. The computer-implemented method also includes inputting, via the processing system, the single complex magnetic resonance image for each slice into a decomposition model trained to predict chemical shifted-like images from the single complex magnetic resonance image for each slice. The computer-implemented method further includes outputting, via the processing system, from the decomposition model the chemical shifted-like images of one or more pre-defined components of the different pre-defined components based on the single complex magnetic resonance image for each slice.

In another embodiment, a system for generating magnetic resonance images is provided. The system includes memory encoding processor-executable routines. The system also includes a processing system including one or more processors and configured to access the memory and to execute the processor-executable routines, wherein the processor-executable routines, when executed by the processing system, cause the processing system to perform actions. The actions include obtaining a single complex magnetic resonance image per slice from magnetic resonance scan data acquired of a subject with a magnetic resonance scanner utilizing a Dixon sequence where signal contributions from water and fat to acquired echo signals are separated, wherein the single complex magnetic resonance image includes a non-zero phase angle between the water and the fat. The actions also include inputting the single complex magnetic resonance image into a decomposition model trained to predict Dixon-like images from the single complex magnetic resonance image. The actions further include outputting from the decomposition model a water image, a fat image, and a field inhomogeneity image based on the single complex magnetic resonance image.

In a further embodiment, a non-transitory computer-readable medium is provided. The non-transitory computer-readable medium including processor-executable code that when executed by a processing system including one or more processors, causes the processing systems to perform actions. The actions include obtaining a single complex magnetic resonance image per slice from magnetic resonance scan data acquired of a subject with a magnetic resonance scanner utilizing a Dixon sequence where signal contributions from water and fat to acquired echo signals are separated, wherein the single complex magnetic resonance image includes a non-zero phase angle between the water and the fat. The actions also include inputting the single complex magnetic resonance image into a decomposition model trained to predict Dixon-like images from the single complex magnetic resonance image. The actions further include outputting from the decomposition model a water image, a fat image, and a field inhomogeneity image based on the single complex magnetic resonance image.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique;

FIG. 2 is a schematic diagram illustrating angles for Dixon MRI;

FIG. 3 is a schematic diagram of a process for generating Dixon-like images via single-shot Dixon scanning, in accordance with aspects of the present disclosure;

FIG. 4 is a flow chart of a method for generating chemical shifted-like images from single-shot scanning (e.g. single shot Dixon scanning), in accordance with aspects of the present disclosure;

FIG. 5 is a schematic diagram of a process for generating synthetic training data, in accordance with aspects of the present disclosure;

FIG. 6 is a schematic diagram of a process for training an AI model to predict Dixon-like images, in accordance with aspects of the present disclosure;

FIG. 7 is a flow chart of a method for training an AI model to generate chemical shifted-like images from single-shot scanning, in accordance with aspects of the present disclosure;

FIG. 8 depicts MR images of a patient's knee derived utilizing the AI model (e.g., utilizing a phase angle of zero degrees versus 180 degrees), in accordance with aspects of the present disclosure;

FIG. 9 depicts MR images of multiple patients' knees derived utilizing the AI model (e.g., utilizing a phase angle of zero degrees versus 180 degrees), in accordance with aspects of the present disclosure;

FIG. 10 depicts MR images of a patient's knee, in accordance with aspects of the present disclosure;

FIG. 11 depicts MR images of a patient's knee (at different orientations) derived utilizing the AI model (e.g., utilizing phase angles of 30 and 180 degrees), in accordance with aspects of the present disclosure;

FIG. 12 depicts Dixon-like images predicted from single-shot Dixon scanning utilizing the AI model of a first patient, in accordance with aspects of the present disclosure;

FIG. 13 depicts Dixon-like images predicted from single-shot Dixon scanning utilizing the AI model of a second patient, in accordance with aspects of the present disclosure; and

FIG. 14 depicts MR images of a patient's knee, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

Deep-learning (DL) approaches discussed herein may be based on artificial neural networks, and may therefore encompass one or more of deep neural networks, fully connected networks, convolutional neural networks (CNNs), vision transformers, unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

The present disclosure provides systems and methods for utilizing artificial intelligence and an MR signal generative model to separate water and fat images in common clinical scenarios using a single MR image (i.e., single complex MR image) with a non-zero phase angle between the water and fat (i.e., single-shot Dixon scanning). In particular, a decomposition model is trained to predict Dixon-like images (e.g., water image, fat image, and field inhomogeneity image (e.g., field map) from a single MR image with the non-zero phase angle between the water and fat. In particular, while the non-zero phase angle is known from the data acquisition to decompose the magnetic resonance complex image, the AI model (i.e., decomposition model) needs to learn the water, fat, and field map per pixel. To train the AI model, the MR signal generative model generates synthetic data to train the AI model. In particular, MR signal generative model is configured to simulate the ground truth (e.g., ground truth water, fat, and field inhomogeneity images) and input (e.g. magnetic resonance complex image) pair (which perfectly one on one matched) using a multi-image Dixon method. Known physical and mathematical definitions are used to generate synthetic training data. In certain embodiments, real data may be utilized as training data depending on the data preparation method.

The disclosed systems and methods, due to one-time Dixon complex scanning (as opposed to multiple point scanning) with just a one-time single recording, provide shorter scan times for patients. In particular, the scanning time can be reduced by a factor of the number of images required by an otherwise Dixon MRI technique. The disclosed systems and methods reduce the complexity of the scanning protocol. The disclosed systems and methods accelerate the daily usage of the MR system. The disclosed systems and methods provide comparable performance to current scanning techniques while reducing the scanning time. The disclosed systems and methods take into account the physical and mathematical definition for the AI model and data scanning to provide richer information with just one scan. By combining prior knowledge with phase control during the scanning, the disclosed systems and methods can efficiently reduce scanning time while predicting the water and fat images as real as possible.

The disclosed embodiments include a system and method for generating magnetic resonance images. The system and method include obtaining, via a processing system including one or more processors, a single complex magnetic resonance image per slice from magnetic resonance scan data acquired of a subject with a magnetic resonance scanner utilizing a chemical-shifted sequence where signal contributions from different predefined components to acquired echo signals are separated, wherein the single complex magnetic resonance image includes a non-zero phase angle between the different predefined components. The system and method also include inputting, via the processing system, the single complex magnetic resonance image for each slice into a decomposition model trained to predict chemical shifted-like images from the single complex magnetic resonance image for each slice. The system and method further include outputting, via the processing system, from the decomposition model the chemical shifted-like images of one or more pre-defined components of the different pre-defined components based on the single complex magnetic resonance image for each slice.

The disclosed embodiments include a system and method for generating magnetic resonance images. The system and method include obtaining, via a processing system including one or more processors, a single complex magnetic resonance image per slice from magnetic resonance scan data acquired of a subject with a magnetic resonance scanner utilizing a Dixon sequence where signal contributions from water and fat to acquired echo signals are separated, wherein the single complex magnetic resonance image includes a non-zero phase angle between the water and the fat. The system and method also include inputting, via the processing system, the single complex magnetic resonance image for each slice into a decomposition model trained to predict Dixon-like images from the single complex magnetic resonance image for each slice. The system and method further include outputting, via the processing system, from the decomposition model a water image, a fat image, and a field inhomogeneity image based on the single complex magnetic resonance image for each slice.

In certain embodiments, the Dixon sequence includes a fast spin echo sequence (or other type of Dixon sequence). In certain embodiments, the magnetic resonance scan data is acquired in a single one-time scan (e.g., utilizing single shot sequence).

In certain embodiments, the decomposition model was trained on synthetic data or real data. In certain embodiments, the system and method include obtaining, via the processing system, magnetic resonance images from other magnetic resonance scan data acquired of one or more other subjects utilizing the chemical shifted sequence (e.g., Dixon sequence), wherein the magnetic resonance images were acquired at different phase angles between the different pre-defined components (e.g., the water and the fat), and the magnetic resonance images include in-phase and out-of-phase images; and utilizing, via the processing system, a trained magnetic resonance signal generative model to generate from the magnetic resonance images both magnetic resonance complex images for different phase angles for the different predefined components (e.g., different water-fat phase angles) for inputting into a deep learning-based model and corresponding ground truth chemical shifted images (e.g., water images, fat images, and field inhomogeneity images). In certain embodiments the system and method include inputting, via the processing system, the magnetic resonance complex images into the deep learning-based model; outputting, via the processing system, respective predicted chemical shifted-like images (e.g., predicted water images, fat images, and field inhomogeneity images) for the magnetic resonance complex images; and utilizing, via the processing system, a loss function based on both the respective predicted chemical shifted-like images (e.g., predicted water images, fat images, and field inhomogeneity images) and the corresponding ground truth chemical-shifted images (e.g., ground truth water images, fast images, and field inhomogeneity images) to train the deep learning-based model to generate the decomposition model. In certain embodiments, the system and method include utilizing, via the processing system, the trained magnetic resonance signal generative model to generate reconstructed magnetic resonance complex images from the respective predicted chemical shifted-like (e.g., predicted water images, fat images, and field inhomogeneity images); and utilizing, via the processing system, a consistency loss function (or other loss function) between the magnetic resonance complex images and the reconstructed magnetic resonance complex images to train the deep learning-based model to generate the decomposition model.

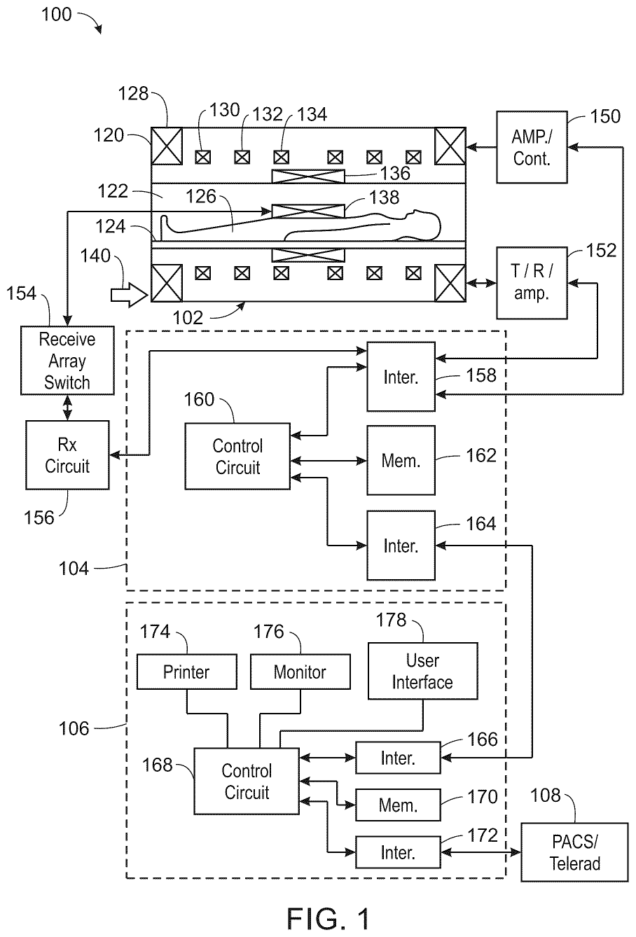

With the preceding in mind, FIG. 1 is a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 to be positioned therein for imaging selected anatomy within the patient.

Scanner 102 includes a series of associated coils for producing controlled magnetic fields for exciting the gyromagnetic material within the anatomy of the subject being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain of the gyromagnetic nuclei within the patient 126 during examination sequences. A radio frequency (RF) coil 136 is configured to generate radio frequency pulses for exciting the certain gyromagnetic nuclei within the patient. In addition to the coils that may be local to the scanner 102, the system 100 also includes a set of receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain of the gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, B0. A power input 44 (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuit 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuit 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuit 104 and system control circuit 106. In certain embodiments, the control and analysis circuit 160, while illustrated as a single unit, may include one or more hardware devices. The system control circuit 106 includes an interface circuit 166, which receives data from the scanner control circuit 104 and transmits data and commands back to the scanner control circuit 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The memory circuit 170 may store a decomposition model trained to decomposition model trained to predict Dixon-like images from a single complex magnetic resonance image having a non-zero phase angle between water and fat. The memory circuit 170 may also store a trained magnetic resonance signal generative model configured to generate synthetic data for training the decomposition model. The programming code may execute one or more algorithms that, when executed by a processing system, are configured to perform processing and reconstruction of acquired data as described below. In certain embodiments, the techniques described herein may occur on a separate computing device having processing circuitry and memory circuitry.

In certain embodiments, the programming code is configured to obtain a single complex magnetic resonance image per slice from magnetic resonance scan data acquired of a subject with a magnetic resonance scanner utilizing a chemical-shifted sequence (e.g., Dixon sequence) where signal contributions from different pre-defined components (e.g., water and fat) to acquired echo signals are separated, wherein the single complex magnetic resonance image includes a non-zero phase angle between the different pre-defined components (e.g., water and the fat). In certain embodiments, the programming code is configured to input the single complex magnetic resonance image for each slice into a decomposition model trained to predict chemical shifted-like (e.g., Dixon-like images) from the single complex magnetic resonance image for each slice. In certain embodiments, the programming code is configured to output from the decomposition model one or more of the chemical shifted-like images for one or more predefined components of the different pre-defined components (e.g., a water image, a fat image, and a field inhomogeneity image) based on the single complex magnetic resonance image for each slice.

In certain embodiments, the chemical-shifted sequence includes a fast spin echo sequence (or other type of Dixon sequence). In certain embodiments, the magnetic resonance scan data is acquired in a single one-time scan (e.g., utilizing single shot sequence).

In certain embodiments, the decomposition model was trained on synthetic data or real data. In certain embodiments, the programming code is configured to obtain magnetic resonance images from other magnetic resonance scan data acquired of one or more other subjects utilizing the chemical-shifted sequence (e.g., Dixon sequence), wherein the magnetic resonance images were acquired at different phase angles between the different predefined components (e.g., water and the fat), and the magnetic resonance images include in-phase and out-of-phase images. In certain embodiments, the programming code is configured to utilize a trained magnetic resonance signal generative model to generate from the magnetic resonance images both magnetic resonance complex images for different phase angles of the different predefined components (e.g., different water-fat phase angles) for inputting into a deep learning-based model and corresponding ground truth chemical-shifted images of the different predefined components (e.g., ground truth water images, fat images, and field inhomogeneity images). In certain embodiments, the programming code is configured to input the magnetic resonance complex images into the deep learning-based model. In certain embodiments, the programming code is configured to output respective predicted chemical-shifted images (e.g., predicted water images, fat images, and field inhomogeneity images) for the magnetic resonance complex images. In certain embodiments, the programming code is configured to utilize a loss function based on both the respective predicted chemical-shifted images (e.g., predicted water images, fat images, and field inhomogeneity images) and the corresponding ground truth chemical-shifted images of the different predefined components (e.g., ground truth water images, fast images, and field inhomogeneity images) to train the deep learning-based model to generate the decomposition model. In certain embodiments, the programming code is configured to utilize the trained magnetic resonance signal generative model to generate reconstructed magnetic resonance complex images from the respective predicted chemical-shifted images (e.g., predicted water images, fat images, and field inhomogeneity images). In certain embodiments, the programming code is configured to utilize a loss function (e.g., consistency loss function) between the magnetic resonance complex images and the reconstructed magnetic resonance complex images to train the deep learning-based model to generate the decomposition model.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

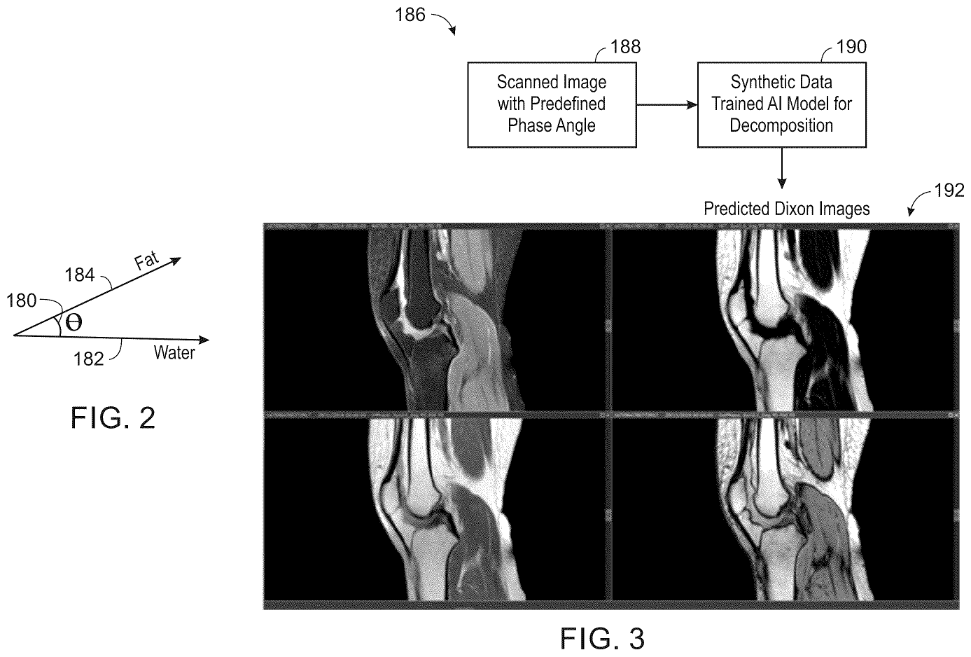

FIG. 2 is a schematic diagram illustrating angles for Dixon MRI. Dixon MRI methods or techniques acquire multiple MR images to separate water and fat images. These methods exploit the frequency differences among the signal peaks from water and fat. As an example, the primary signal peaks of fat and water in MRI resonate at 3.5 parts per million (ppm) frequency difference which is 224 hertz (Hz) at 1.5 Tesla (T). The frequency difference leads to relative phase accrual (where the MR signal is complex valued) between the water and fat signals during MR signal excitation. Multiple MR images are typically acquired over multiple repetition times or over fewer relatively longer repetition times leading to long acquisition time. For example, traditional 2-point or 3-point scanning uses multiple MR images with specified phase differences between water and fat images.

Dixon MRI involves acquiring multiple MRI images with different phase angles 180 (e.g., θ) between water (indicated by reference numeral 182) and fat (indicated by reference numeral 184). Some of the common images acquired are in-phase images where the phase angle, θ, is zero degrees and out-of-phase images where the phase angle, θ, is 180 degrees.

FIG. 3 is a schematic diagram of a process 186 for generating Dixon-like images via single-shot Dixon scanning. The process 186 includes obtaining (e.g., generating) a single MR image 188 (e.g., complex MR image) of a subject with a predefined phase angle between fat and water. The single MR image 188 is generated from magnetic resonance scan data acquired of a subject with a magnetic resonance scanner (e.g., scanner 102 in FIG. 1) utilizing a Dixon sequence where signal contributions from water and fat to acquired echo signals are separated. In certain embodiments, the Dixon sequence may be a fast spin echo or triple echo Dixon sequence (or other type of Dixon sequence). The magnetic resonance scan data is acquired in a single one-time scan or recording (e.g., utilizing single shot sequence).

The single MR image 188 is inputted into a trained decomposition model 190. The trained decomposition model 190 is an AI model (e.g., deep learning-based model) trained with synthetic data as described in greater detail below. The decomposition model 190 is trained to decompose the Dixon complex scanning (i.e., the single MR image 188) and to predict Dixon-like images 192 from the single MR image 188. The predicted Dixon-like images are outputted from the trained decomposition model 190. The Dixon-like images 192 may include a fat image, a water, a field inhomogeneity image (e.g., field map), an in-phase image, and/or an out-of-phase image.

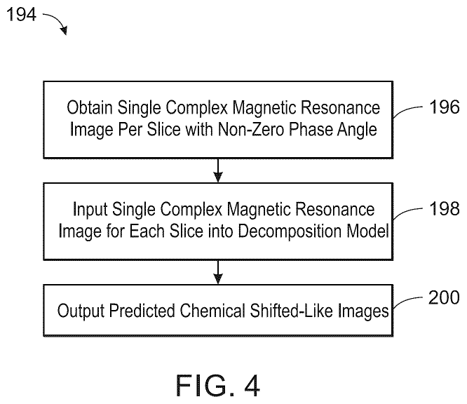

FIG. 4 is a flow chart of a method 194 for generating Dixon-like images from single-shot scanning (e.g., single shot Dixon scanning). One or more steps of the method 194 may be performed by processing circuitry of the magnetic resonance imaging system 100 in FIG. 1 or a remote computing system. One or more steps of the method 194 may be performed simultaneously or in a different order from that depicted in FIG. 4.

The method 194 includes obtaining (e.g., generating) a single complex magnetic resonance image per slice from magnetic resonance scan data acquired of a subject with a magnetic resonance scanner (e.g., scanner 102 in FIG. 1) utilizing a chemical shifted sequence (e.g., Dixon sequence) where signal contributions from different pre-defined components (e.g., water and fat) to acquired echo signals are separated, wherein the single complex magnetic resonance image has a non-zero phase angle between the different pre-defined components (e.g., water and the fat) (block 196). In certain embodiments, the chemical shifted sequence may be a fast spin echo (or other type of chemical shifted or Dixon sequence). The magnetic resonance scan data is acquired in a single one-time scan or recording (e.g., utilizing single shot sequence). The method 194 also includes inputting the single complex magnetic resonance image (for each slice) into a decomposition model trained to predict chemical shifted-like images (e.g., Dixon-like images) from the single complex magnetic resonance image for each slice (block 198). The method 194 further includes outputting from the decomposition model the chemical shifted-like (e.g., Dixon-like images) based on the single complex magnetic resonance image for each slice (block 200). The chemical shifted like images may be for the different pre-defined components. In certain embodiments, the chemical shifted-like (e.g., Dixon-like images) may include a fat image, a water image, a field inhomogeneity image (e.g., field map), an in-phase image, and/or an out-of-phase image.

In certain embodiments, the training of the AI model may be with real data depending on the data preparation method. In certain embodiments, synthetic data is utilized for training the AI model. An MR signal generative model is utilized for generating the synthetic data for training the AI model. The MR signal generative model for complexed value MR image (x,y) with phase angle θ between water and fat and in the presence of field inhomogeneity, Field Map, is given by the following equation:

$$Image ( x , y ) = ( Water ( x , y ) + Fat ( x , y ) {e}^{j θ} ) {e}^{j F i e l d M a p ( x , y )} . \qquad ( 1 )$$

For the AI-based water, fat, and field map separation from a single image, the parameter θ (i.e., phase angle between water and fat) is known from the data acquisition for decomposing the magnetic resonance complex image. The AI model needs to learn (i.e., be trained) the water, fat, and field map per pixel. Suppose the AI model is F(·), then

$$water ’ , fat ’ , fieldmap ’ = F ( image , θ ) , \qquad ( 2 )$$

where water′, fat′, and fieldmap′ are an estimated real-value matrix for water/fat intensity and background phase error due field map inhomogeneities in radians. The object function for training is the following:

$$Loss = abs ( water - {water}^{′} ) + ab s ( fat - {fat}^{′} ) + abs ( fieldmap - {fieldmap}^{′} ) + abs ( image - ( {water}^{′} + {fat}^{′ {e}^{j θ}} ) {e}^{{jFieldMap}^{′}} ) . \qquad ( 3 )$$

Specifically, due to the signal generation function in Equation 1, the ground truth and input pair can be simulated by the formular and given θ from the current Dixon product pipeline (i.e., one-shot Dixon scanning). Therefore, the training of the AI model can be done synthetically by using knowledge of the definition. It should be noted that any type of loss function can be utilized for the training.

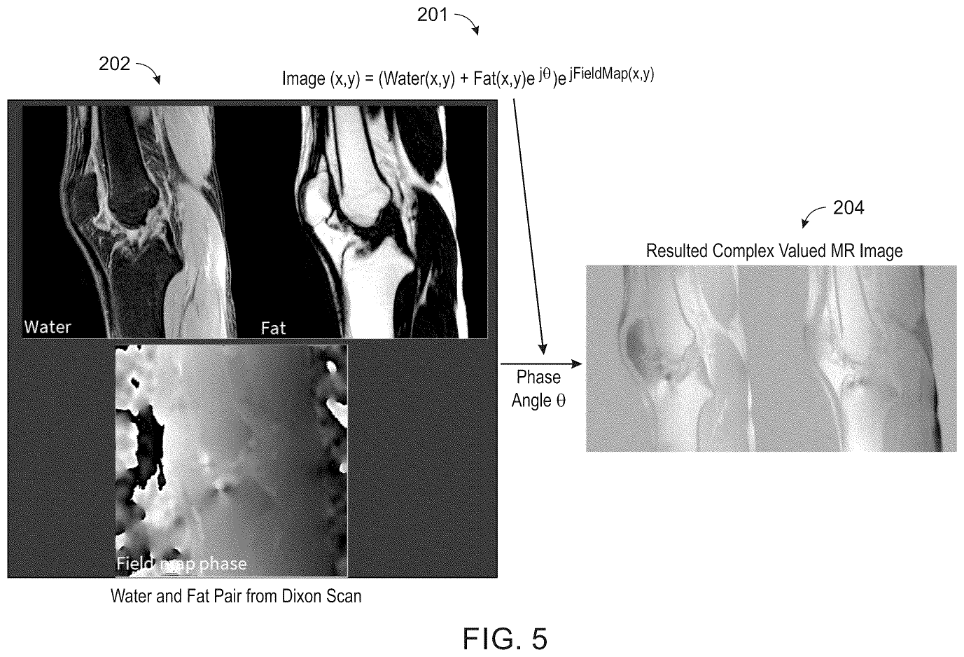

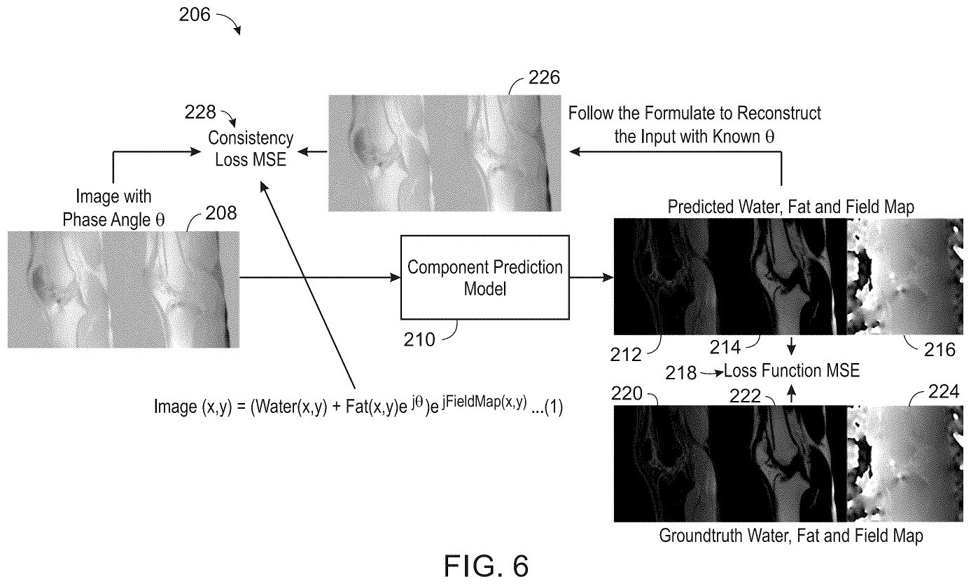

FIG. 5 is a schematic diagram of a process 201 for generating synthetic training data. Synthetic data for the training of the AI model (i.e., decomposition model) is generated using a multi-image Dixon method. Known physical and mathematical definitions (e.g., Equation 1) are used to generate the synthetic training data. As depicted in FIG. 5, magnetic resonance images 202 (e.g., field map phase and water and fat pair) from other magnetic resonance scan data acquired of one or more other subjects utilizing a Dixon sequence are obtained (e.g., generated), wherein the magnetic resonance images were acquired at different phase angles between the water and the fat. These magnetic resonance images may include in-phase and out-of-phase images. These images 202 along with Equation 1 (i.e., MR signal generative model) and a given phase angle θ are utilized to generate a complex MR image 204 (e.g., as an input image) and a ground truth water, fat, and field map images (see FIG. 6) (i.e., perfectly one on one matched ground truth and input pair).

FIG. 6 is a schematic diagram of a process 206 for training an AI model to predict Dixon-like images. In certain embodiments, the AI model is trained to predict Dixon-like images for a single given phase angle. In certain embodiments, the AI model is trained to predict Dixon-like images for different given phase angles. The process 206 includes inputting an MR image 208 (e.g., complex MR image) having a given phase angle θ into a deep learning-based model 210 (e.g., referred to as a component prediction model or a decomposition model). The component prediction model 210 predicts a water image 212, a fat image 214, and a field map 216 from the MR image 208. The process 206 includes determining a loss function 214 (e.g., mean squared error (MSE)) between the predicted water image 214, fat image 216, and field map 218 and compare it to the ground truth water image 220, ground truth fat image 222, and ground truth field map 224 that correspond to the inputted MR image 208. The loss function 214 is utilized to further train the model 210. The process 206 also includes generating a reconstructed MR image 226 (e.g., reconstructed complex MR image) from the predicted water image 212, fat image 214, and field map 216 utilizing Equation 1 and the given phase θ. The process 206 includes determining a consistency loss 228 (e.g., MSE) between the MR image 208 and the reconstructed MR image 226 (e.g., utilizing Equation 1). The consistency loss 228 is utilized to further train the model 210.

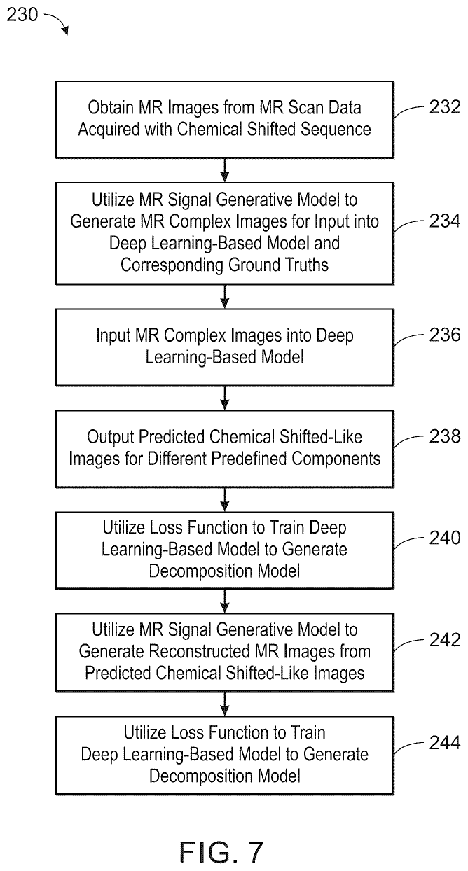

FIG. 7 is a flow chart of a method 230 for training an AI model to generate chemical shifted-like (e.g., Dixon-like images) from single-shot scanning (e.g., single-shot Dixon scanning). One or more steps of the method 230 may be performed by processing circuitry of the magnetic resonance imaging system 100 in FIG. 1 or a remote computing system. One or more steps of the method 230 may be performed simultaneously or in a different order from that depicted in FIG. 7.

The method 230 includes obtaining magnetic resonance images from other magnetic resonance scan data acquired of one or more other subjects utilizing the chemical-shifted sequence (e.g., Dixon sequence) (block 232). In certain embodiments, chemical shifted sequence may be a fast spin echo e (or other type of Dixon sequence). The magnetic resonance images were acquired at different phase angles between the different pre-defined components (e.g., water and the fat). The magnetic resonance images may include in-phase and out-of-phase images.

The method 230 also includes utilizing a trained magnetic resonance signal generative model to generate from the magnetic resonance images both MR complex images for different phase angles for the different pre-defined components (e.g., different water-fat phase angles) for inputting into a deep learning-based model and corresponding ground truth images for the different pre-defined components (e.g., ground truth water images, fat images, and field inhomogeneity images) (block 234). The method 230 further includes inputting the MR complex images into the deep learning-based model (block 236). The method 230 even further includes outputting respective predicted chemical shifted-like images for the different pre-defined components (e.g., predicted water images, fat images, and field inhomogeneity images) for the MR complex images (block 238).

The method 230 also includes utilizing a loss function based on both the respective predicted chemical shifted-like images for the different pre-defined components (e.g., predicted water images, fat images, and field inhomogeneity images) and the corresponding ground truth images for the different pre-defined components (e.g., ground truth water images, fast images, and field inhomogeneity images) to train the deep learning-based model to generate the decomposition model (block 240). The method 230 further includes utilizing the trained magnetic resonance signal generative model to generate reconstructed MR complex images from the respective predicted chemical shifted-like images for the different pre-defined components (e.g., predicted water images, fat images, and field inhomogeneity images) (block 242). The method 230 even further includes utilizing a loss function (e.g., consistency loss function or other loss function) between the MR complex images and the reconstructed MR complex images to train the deep learning-based model to generate the decomposition model (block 244).

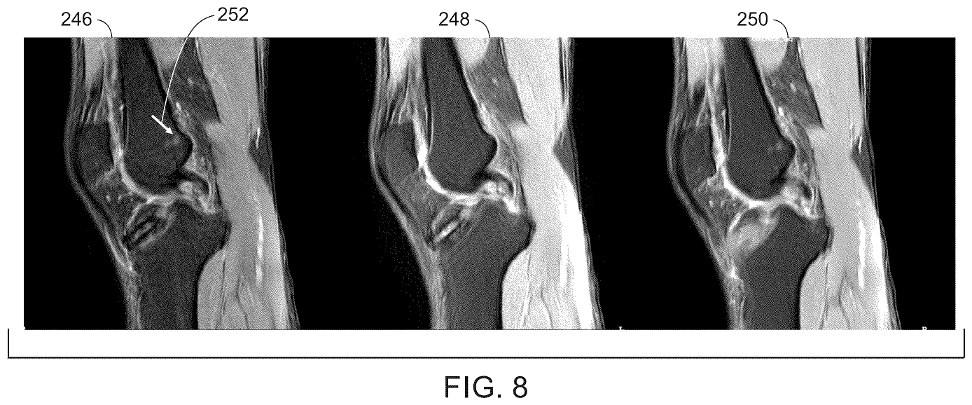

FIGS. 8-14 disclose testing of the disclosed techniques. Testing utilized a patient population. In particular, 38 patients clinically requiring knee MRI were scanned in an institutional review board approved study on a research 0.5T MR scanner and a product 1.5T MR scanner. 3-point fast triple echo Dixon (FTED) based acquisition was done with sagittal, coronal, and axial orientations for each of the patients. Synthetic MR image generation was utilized for training and testing. Water, fat, field map images generated in the product Dixon post processing of each of the FTED acquired data were saved. FTED based data acquisition included three MR images with water and fat having angles of 180 degrees, zero degrees, and negative 180 degrees between them. The MR signal generation model (i.e., Equation 1) was used to generate input images to the deep learning-based model at various θ. The water, fat, and field map images were used to generate the output (i.e., MR complex image) of the deep learning-based model. The loss function in Equation 3 was utilized to train the deep learning-based model. Each of the 38 patients have three orientations. Of these, 29 patients were used for training and 9 patient were used for validation. In the following, a θ of zero degrees (i.e., in-phase), 30 degrees, and 180 degrees (i.e., out-of-phase) were used for accessing the results.

FIG. 8 depicts MR images of a patient's knee derived utilizing the AI model (e.g., utilizing a phase angle of zero degrees versus 180 degrees). MR images 246, 248, 250 are sagittal MR images of a knee acquired with FTED. MR image 246 is a ground truth water image derived via typical Dixon processing. MR image 248 is an AI processed (utilizing the AI model or decomposition model disclosed herein) water image derived when the input image was an in-phase image (phase angle of zero degrees). MR image 250 is an AI processed (utilizing the AI model or decomposition model disclosed herein) water image derived when the input image was an out-of-phase image (phase angle of 180 degrees). Although the in-phase MR image with a phase angle of zero degrees theoretically as minimal information present about water and fat content at each pixel/voxel, the AI model could generate realistic looking water images. The pathology of bone edema (represented by arrow 252) marked by hyperintensity in the bone in the AI generated water images is present when the input MR image has a phase angle of 180 degrees. This gives an indication that AI can generate water images when the input image has the information.

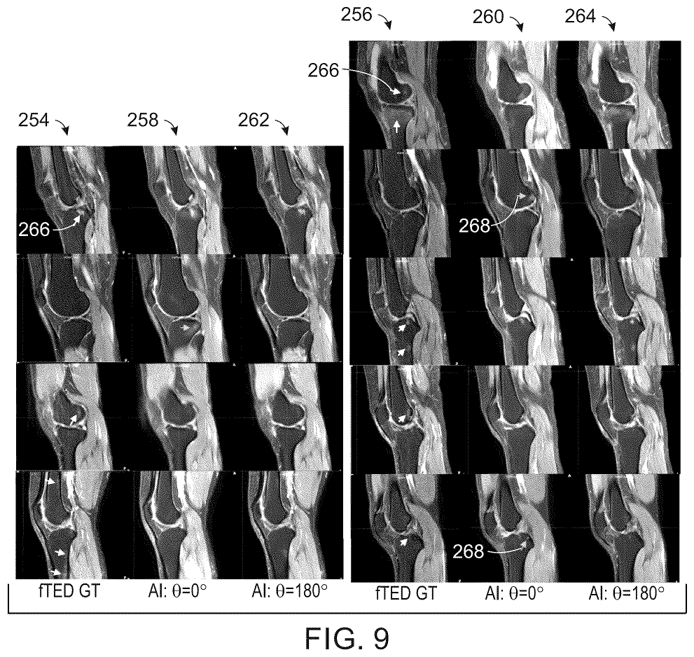

FIG. 9 depicts MR images of multiple patients' knees (nine patents) derived utilizing the AI model (e.g., utilizing a phase angle of zero degrees versus 180 degrees). The MR images are sagittal MR images of a knee acquired with FTED. Columns 254, 256 of MR images are ground truth water images derived via typical Dixon processing. Columns 258, 260 of MR images are AI processed (utilizing the AI model or decomposition model disclosed herein) water images derived when the input image was an in-phase image (phase angle of zero degrees). Columns 262, 264 of MR images are AI processed (utilizing the AI model or decomposition model disclosed herein) water images derived when the input image was an out-of-phase image (phase angle of 180 degrees). Pathology in the ground truth images are marked by arrows 266. The few false pathologies are marked by arrows 268 and are only observed in the AI generated water images where the input image had a phase angle of zero degrees. This is a reason to keep input images at a non-zero phase angle as disclosed above. Improved SNR and fewer motion artifacts were observed in the AI generated water images. FIGS. 8 and 9 demonstrate the importance of the phase angle (i.e., zero versus 180 degrees).

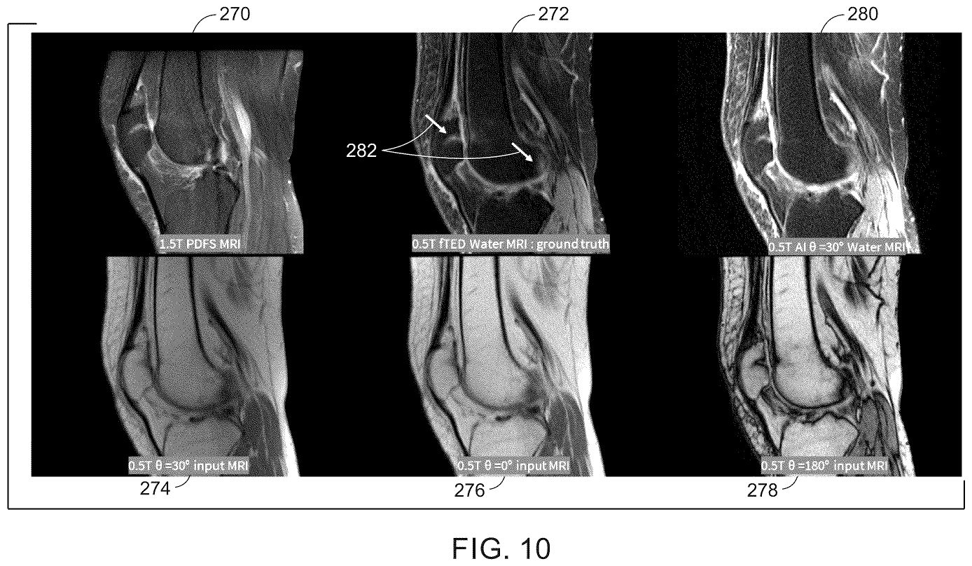

FIG. 10 depicts MR images of a patient's knee. MR image 270 is a proton density fat saturated (PDFS) MR image acquired at 1.5T. MR image 272 is a ground truth water image acquired with FTED at 0.5T and derived via typical Dixon processing. MR image 274 is an input image into the AI model acquired with FTED at 0.5T and having a phase angle of 30 degrees. MR image 276 is an input image into the AI model acquired with FTED at 0.5T and having a phase angle of zero degrees. MR image 278 is an input image into the AI model acquired with FTED at 0.5T and having a phase angle of 180 degrees. MR images 2774, 276, 278 are also PDFS images. MR image 280 is an AI processed (utilizing the AI model or decomposition model disclosed herein) water image derived when the input image has a phase angle of 30 degrees (i.e. MR image 274). The water image generated from an input image having a phase angle 30 degrees was able to detect the pathology (marked by arrows 282 on MR image 272). As depicted, the MR image 274 appears like MR image 276 when viewed as a typical MR image (i.e., magnitude valued images in DICOM).

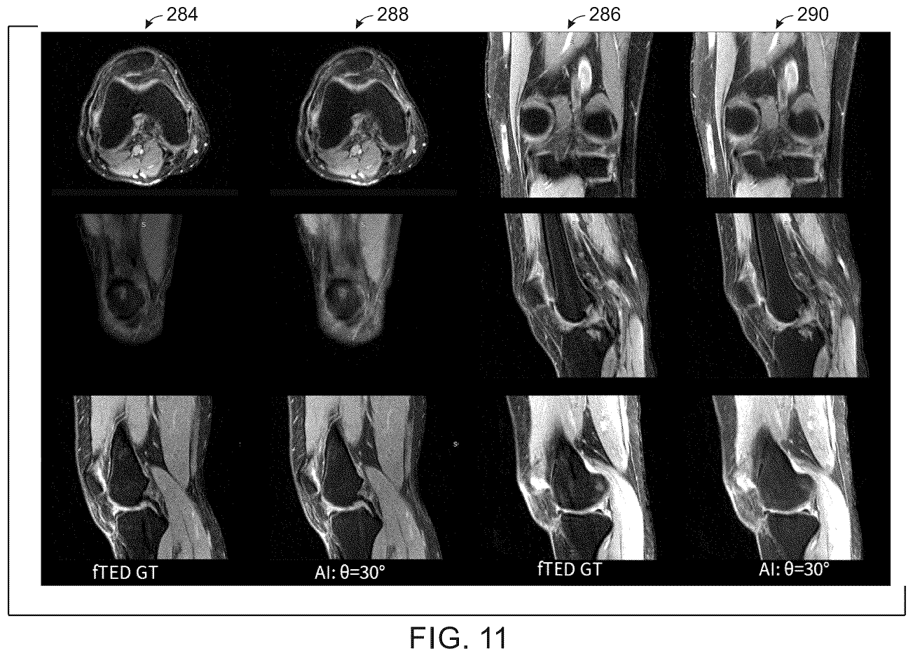

FIG. 11 depicts MR images of a patients' knees derived utilizing the AI model (e.g., utilizing phase angles of zero and 30 degrees). Columns 284 and 286 of MR images are ground truth water images generated using FTED acquisition and processing. Columns 288 and 290 of MR images are corresponding AI processed (utilizing the AI model or decomposition model disclosed herein) water images derived when the input image has a phase angle of 30 degrees. The MR images vary in orientation. The AI processed water images are similar to the ground truth water images.

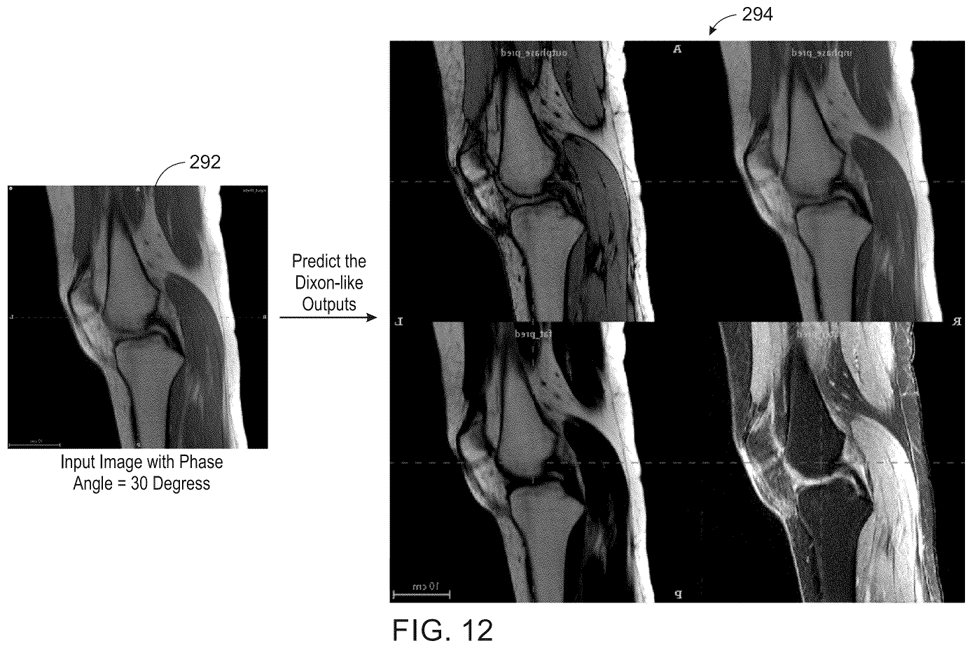

FIG. 12 depicts Dixon-like images predicted from single-shot Dixon scanning utilizing the AI model of a first patient. The left side of FIG. 12 depicts an input MR image 292 of a knee having a phase angle of 30 degrees. A magnitude image is shown for the input MR image 292. However, the single complex MR image is inputted into the AI model and Dixon-like MR images 294 are outputted from the AI model.

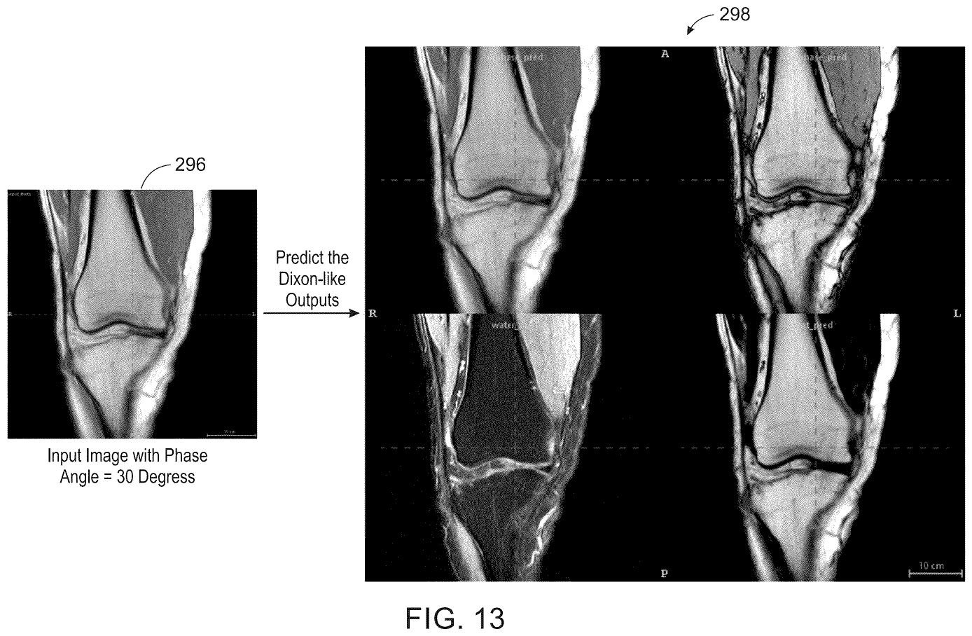

FIG. 13 depicts Dixon-like images predicted from single-shot Dixon scanning utilizing the AI model of a second patient. The left side of FIG. 13 depicts an input MR image 296 of a knee having a phase angle of 30 degrees. A magnitude image is shown for the input MR image 296. However, the single complex MR image is inputted into the AI model and Dixon-like MR images 298 are outputted from the AI model.

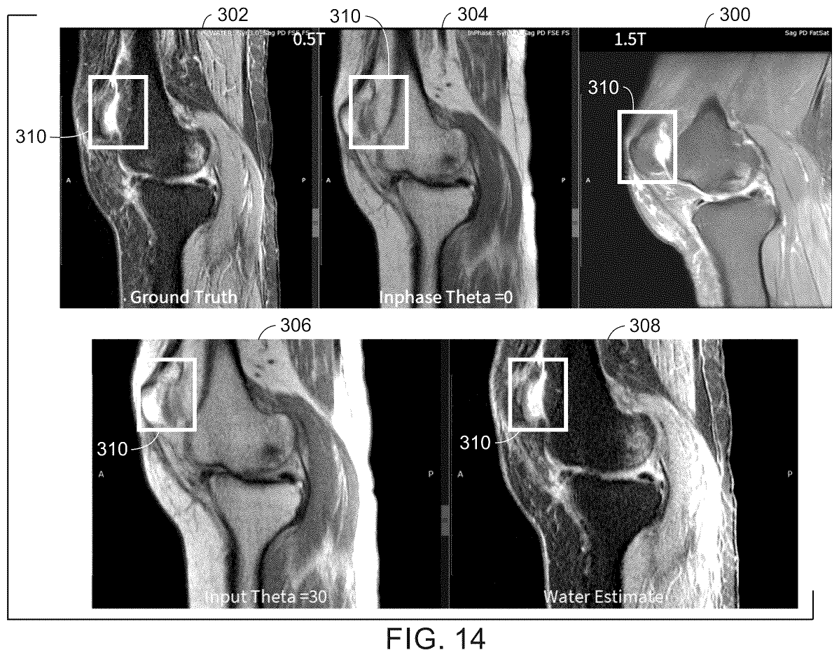

FIG. 14 depicts MR images of a patient's knee. MR image 300 is a proton density fat saturated (PDFS) MR image acquired at 1.5T. MR image 302 is a ground truth water image acquired with FTED at 0.5T and derived via typical Dixon processing. MR image 304 was acquired at FTED at 0.5T and has a phase angle of zero degrees. MR image 306 is an input image into the AI model acquired with FTED at 0.5T and having a phase angle of 30 degrees. MR image 308 is an AI processed (utilizing the AI model or decomposition model disclosed herein) water image derived when the input image has a phase angle of 30 degrees (i.e. MR image 306). The water image generated from an input image having a phase angle 30 degrees was able to detect the pathology (edema marked by box 310). The water within the box 310 in the MR image 308 can be estimated correctly (as opposed to a water image derived from an input image having a phase angle of zero degrees). MR image 308 has meaningful structure similar to MR image 302. The MR image 306 looks similar to MR image 304.

Technical effects of the disclosed subject matter include providing techniques that utilize artificial intelligence and an MR signal generative model to separate water and fat images in common clinical scenarios using a single MR image (i.e., single complex MR image) with a non-zero phase angle between the water and fat (i.e., single-shot Dixon scanning). Technical effects of the disclosed subject matter include, due to one-time Dixon complex scanning (as opposed to multiple point scanning) with just a one-time single recording, providing shorter scan times for patients. In particular, the scanning time can be reduced by a factor of the number of images required by an otherwise Dixon MRI technique. Technical effects of the disclosed subject matter include reducing the complexity of the scanning protocol. Technical effects of the disclosed subject matter include accelerating the daily usage of the MR system. Technical effects of the disclosed subject matter include providing comparable performance to current scanning techniques while reducing the scanning time.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for generating magnetic resonance images:
obtaining, via a processing system comprising one or more processors, a single complex magnetic resonance image per slice from magnetic resonance scan data acquired of a subject with a magnetic resonance scanner utilizing a chemical shifted sequence where signal contributions from different pre-defined components to acquired echo signals are separated, wherein the single complex magnetic resonance image comprises a non-zero phase angle between the different pre-defined components;
inputting, via the processing system, the single complex magnetic resonance image into a decomposition model trained to predict chemical shifted-like images from the single complex magnetic resonance image; and
outputting, via the processing system, from the decomposition model the chemical shifted-like images of one or more pre-defined components of the different pre-defined components based on the single complex magnetic resonance image.

2. The computer-implemented method of claim 1, wherein the chemical shifted sequence comprises a Dixon sequence.

3. The computer-implemented method of claim 2, wherein the Dixon sequence comprises a fast spin echo sequence.

4. The computer-implemented method of claim 1, wherein the decomposition model was trained on synthetic data or real data.

5. The computer-implemented method of claim 1, further comprising:
obtaining, via the processing system, magnetic resonance images from other magnetic resonance scan data acquired of one or more other subjects utilizing the chemical shifted sequence, wherein the magnetic resonance images were acquired at different phase angles between the different pre-defined components, and the magnetic resonance images comprise in-phase and out-of-phase images; and
utilizing, via the processing system, a trained magnetic resonance signal generative model to generate from the magnetic resonance images both magnetic resonance complex images for different phase angles between the different pre-defined components for inputting into a deep learning-based model and corresponding ground truth images of the different pre-defined components.

6. The computer-implemented method of claim 5, further comprising:
inputting, via the processing system, the magnetic resonance complex images into the deep learning-based model;
outputting, via the processing system, respective predicted chemical shifted-like images for the one or more pre-defined components of the different pre-defined components for the magnetic resonance complex images; and
utilizing, via the processing system, a loss function based on both the respective predicted chemical shifted-like images and the corresponding ground truth images of the different pre-defined components to train the deep learning-based model to generate the decomposition model.

7. The computer-implemented method of claim 6, further comprising:
utilizing, via the processing system, the trained magnetic resonance signal generative model to generate reconstructed magnetic resonance complex images from the respective predicted chemical shifted-like images; and
utilizing, via the processing system, a consistency loss function between the magnetic resonance complex images and the reconstructed magnetic resonance complex images to train the deep learning-based model to generate the decomposition model.

8. The computer-implemented method of claim 1, wherein the different pre-defined components comprise water and fat.

9. A system for generating magnetic resonance images, comprising:
a memory encoding processor-executable routines;
a processing system comprising one or more processors and configured to access the memory and to execute the processor-executable routines, wherein the processor-executable routines, when executed by the processing system, cause the processing system to:
obtain a single complex magnetic resonance image per slice from magnetic resonance scan data acquired of a subject with a magnetic resonance scanner utilizing a Dixon sequence where signal contributions from water and fat to acquired echo signals are separated, wherein the single complex magnetic resonance image comprises a non-zero phase angle between the water and the fat;
input the single complex magnetic resonance image for each slice into a decomposition model trained to predict Dixon-like images from the single complex magnetic resonance image for each slice; and
output from the decomposition model a water image, a fat image, and a field inhomogeneity image based on the single complex magnetic resonance image for each slice.

10. The system of claim 9, wherein the Dixon sequence comprises a fast spin echo sequence.

11. The system of claim 9, wherein the decomposition model was trained on synthetic data or real data.

12. The system of claim 9, wherein the processor-executable routines, when executed by the processing system, further cause the processing system to:
obtain magnetic resonance images from other magnetic resonance scan data acquired of one or more other subjects utilizing the Dixon sequence, wherein the magnetic resonance images were acquired at different phase angles between the water and the fat, and the magnetic resonance images comprise in-phase and out-of-phase images; and
utilize a trained magnetic resonance signal generative model to generate from the magnetic resonance images both magnetic resonance complex images for different water-fat phase angles for inputting into a deep learning-based model and corresponding ground truth water images, fat images, and field inhomogeneity images.

13. The system of claim 12, wherein the processor-executable routines, when executed by the processing system, further cause the processing system to:
input the magnetic resonance complex images into the deep learning-based model;
output respective predicted water images, fat images, and field inhomogeneity images for the magnetic resonance complex images; and
utilize a loss function based on both the respective predicted water images, fat images, and field inhomogeneity images and the corresponding ground truth water images, fast images, and field inhomogeneity images to train the deep learning-based model to generate the decomposition model.

14. The system of claim 13, wherein the processor-executable routines, when executed by the processing system, further cause the processing system to:
utilize the trained magnetic resonance signal generative model to generate reconstructed magnetic resonance complex images from the respective predicted water images, fat images, and field inhomogeneity images; and
utilize a consistency loss function between the magnetic resonance complex images and the reconstructed magnetic resonance complex images to train the deep learning-based model to generate the decomposition model.

15. A non-transitory computer-readable medium, the computer-readable medium comprising processor-executable code that when executed by a processing system comprising one or more processors, causes the processing system to:
obtain a single complex magnetic resonance image per slice from magnetic resonance scan data acquired of a subject with a magnetic resonance scanner utilizing a Dixon sequence where signal contributions from water and fat to acquired echo signals are separated, wherein the single complex magnetic resonance image comprises a non-zero phase angle between the water and the fat;
input the single complex magnetic resonance image for each slice into a decomposition model trained to predict Dixon-like images from the single complex magnetic resonance image for each slice; and
output from the decomposition model a water image, a fat image, and a field inhomogeneity image based on the single complex magnetic resonance image for each slice.

16. The non-transitory computer-readable medium of claim 15, wherein the decomposition model was trained on synthetic data or real data.

17. The non-transitory computer-readable medium of claim 15, wherein the processor-executable code, when executed by the processing system, further causes the processing system to:
obtain magnetic resonance images from other magnetic resonance scan data acquired of one or more other subjects utilizing the Dixon sequence, wherein the magnetic resonance images were acquired at different phase angles between the water and the fat, and the magnetic resonance images comprise in-phase and out-of-phase images; and
utilize a trained magnetic resonance signal generative model to generate from the magnetic resonance images both magnetic resonance complex images for different water-fat phase angles for inputting into a deep learning-based model and corresponding ground truth water images, fat images, and field inhomogeneity images.

18. The non-transitory computer-readable medium of claim 17, wherein the processor-executable code, when executed by the processing system, further causes the processing system to:
input the magnetic resonance complex images into the deep learning-based model;
output respective predicted water images, fat images, and field inhomogeneity images for the magnetic resonance complex images; and
utilize a loss function based on both the respective predicted water images, fat images, and field inhomogeneity images and the corresponding ground truth water images, fast images, and field inhomogeneity images to train the deep learning-based model to generate the decomposition model.

19. The non-transitory computer-readable medium of claim 18, wherein the processor-executable code, when executed by the processing system, further causes the processing system to:
utilize the trained magnetic resonance signal generative model to generate reconstructed magnetic resonance complex images from the respective predicted water images, fat images, and field inhomogeneity images; and
utilize a consistency loss function between the magnetic resonance complex images and the reconstructed magnetic resonance complex images to train the deep learning-based model to generate the decomposition model.

20. The non-transitory computer-readable medium of claim 15, wherein the Dixon sequence comprises a fast spin echo sequence.
